(19) United States
(12) Patent Application Publication
(10) Pub. No.: US 2016/0338777 A1
(43) Pub. Date: Nov. 24, 2016

(54) System and Method for Precision Position Detection and Reproduction During Surgery

(71) Applicant: Radlink, Inc., El Segundo, CA (US)

(72) Inventors: Brad L. Penenberg, Los Angeles, CA (US); Wenchao Tao, Los Angeles, CA (US)

(73) Assignee: Radlink, Inc., El Segundo, CA (US)

(21) Appl. No.: 15/153,209

(22) Filed: May 12, 2016

Related U.S. Application Data
(60) Provisional application No. 62/164,347, filed on May 20, 2015.

(51) Int. Cl. A61B 34/10; G06T 11/60; A61B 6/00; A61B 90/00; A61B 5/00; G06T 13/80; A61B 5/11

(52) U.S. Cl. CPC A61B 34/10; G06T 13/80; G06T 11/60; A61B 5/11; A61B 90/37; A61B 5/7278; A61B 6/50; A61B 6/463; A61B 5/743; A61B 5/7405; A61B 5/746; G06T 2200/24; G06T 2210/41; A61B 2562/0219; A61B 2034/105; A61B 2034/102

(57) ABSTRACT

A computerized visual orientation surgery assist system and method receives initial anatomic image information of a patient scan taken at a registration position of the patient; receives initial positional information from a sensor positioned on the patient at a registration position, where the positional sensor senses spatial position in three dimensions and transmits the positional information; establishes the initial positional information as an origin in three-dimensional space for the initial anatomic image information; displays a visual representation of the initial anatomic image information on a computerized display; receives subsequent positional information from the sensor associated with movement of the patient; and updates the computerized display to reflect the subsequent positional information with respect to the initial positional information.

### CROSS REFERENCE TO RELATED APPLICATIONS

The current application claims priority to U.S. Provisional Application Ser. No. 62/164,347, filed May 12, 2015, the entire disclosure of which is incorporated herein by reference.

### TECHNICAL FIELD

This disclosure relates to an apparatus, system and associated method for sensing and displaying positional and orientation image information associated with surgical procedures.

### BACKGROUND

Patients are exposed to a series of x-ray radiation during certain types of surgery, such as total hip arthroplasty because of the requirement that the patient be placed in a desired position (i.e. orientation), moved around, and returned to that desired position during surgery. Repeated x-rays may be taken to assure that, after the patient had been moved, the patient is returned to the desired position to complete surgery.

In addition, during total hip arthroplasty, the cup position as measured on radiographic X-ray image is not accurate when the patient's pelvis is tilted (Sagittal Plane) and/or rotated (Transverse plane). Adjustment factors are needed to compensate the non-ideal patient position, such that no additional X-rays are required to derive more accurate measurements for both abduction and anteversion.

Therefore, there is a need for a new surgical system and associated techniques that improve the process of certain surgical procedures and may also reduce the number of x-rays or other imaging scans that need of the patient to be taken and improve the accuracy of the desired position of the patient for the surgery. There is a need for a new surgical system and associated techniques that better allow a surgeon to visualize the position of the patient's anatomy and/or visualize the position of various surgical tools, implants, procedural steps as the patient is being moved during surgery.

### SUMMARY

The current disclosure provides a system and method that may be useful to minimize a patient's exposure to X-rays during surgery, such as total hip arthroplasty. During surgery, an orientation sensor mounted onto the patient and/or onto a surgical tool or implant during surgery may monitor, transmit and/or record movement of the patient that is reflected on a display visible to a surgeon (or other practitioner) so that, for example, the patient can return to a desired orientation at any time during surgery. In addition, adjustment factors can be calculated and displayed to account for a tilted or rotated anatomical items, surgical tools, implants and/or procedural steps as the patient is moved during surgery.

An aspect of the current disclosure is directed to a visual orientation surgery assist system that includes a positional sensor sensing spatial position in three dimensions and transmitting positional information in three dimensions; and a computerized display system having a display, a receiver receiving the positional information from the positional sensor, a microcontroller operatively coupled to the receiver and to the display and having access to system memory, where the system memory includes software instructions causing the CPU to perform the steps of (in no particular order): receiving initial anatomic image information of a patient scan taken at a registration position of the patient; receiving initial positional information from the sensor positioned on the patient at the registration position; establishing the initial positional information as an origin in three-dimensional space for the initial anatomic image information; displaying an visual representation of the initial anatomic image information on the display; receiving subsequent positional information from the sensor associated with movement of the patient; and updating the display to reflect the subsequent positional information with respect to the initial positional information. In an embodiment, the positional sensor includes a triple-axis gyrometer, a triple-axis accelerometer, and a triple-axis magnetometer. In a more detailed embodiment, the positional sensor further includes a computing component programmed with a fusion algorithm that combines outputs of the triple-axis gyrometer, the triple-axis accelerometer, and the triple-axis magnetometer into positional information comprising pitch, yaw and roll information. Alternatively, or in addition, the positional information transmitted by the positional sensor includes pitch, yaw and roll information.

In an embodiment, the patient scan includes an x-ray scan. In a more detailed embodiment, the visual representation of the initial anatomic image information on the display includes x-ray scan images. In a further detailed embodiment, the subsequent positional information updated to the display includes tilt and rotation information overlayed with the visual representation of the initial anatomic image information. Alternatively, or in addition, the subsequent positional information updated to the display includes translational information with respect to the origin overlayed with the visual representation of the initial anatomic image information. Alternatively, or in addition, the software instructions cause the CPU to perform the additional step of providing at least one of a visual and an audible notification when the subsequent positional information updated to the display reaches a predetermined proximity to the origin. Alternatively, or in addition, the subsequent positional information updated to the display includes reference lines reflecting updated orientations for surgical procedural steps overlayed with the visual representation of the initial anatomic image information. Alternatively, or in addition, the subsequent positional information updated to the display includes reference ellipses reflecting updated orientations for surgical procedural steps overlayed with the visual representation of the initial anatomic image information.

In an embodiment, the visual representation of the initial anatomic image information on the display includes an animated virtual representation of an anatomical body part associated with the location of the positional sensor on the patient's anatomy. In further detailed embodiment, the subsequent positional information updated to the display includes animation of the virtual representation of the anatomical body part. In yet a further detailed embodiment, the animation of the virtual representation of the anatomical body part includes animations representing movement of the anatomical body part in three-dimensional space. In a yet a further detailed embodiment, the animation of the virtual representation of the anatomical body part includes two-dimensional animations representing movement of the anatomical body part in three-dimensional space. Alternatively, or in addition, the animation of the virtual representation of the anatomical body part includes an animated representation of a surgical implant implanted thereto. Alternatively, or in addition, the animation of the virtual representation of the anatomical body part includes an animated representation of a surgical tool associated therewith. Alternatively, or in addition, the animation of the virtual representation of the anatomical body part includes a representation of surgical steps to be performed with respect to the anatomical body part. Alternatively, or in addition, the representation of surgical steps to be performed with respect to the anatomical body part is an animated representation of surgical steps that represent movement of aspects of the surgical step in three-dimensional space as the anatomical body part is moved.

Another aspect of the current disclosure is directed to a computerized visual orientation surgery assist method that includes the steps of: receiving initial anatomic image information of a patient scan taken at a registration position of the patient; receiving initial positional information from a sensor positioned on the patient at a registration position, where the positional sensor senses spatial position in three dimensions and transmits the positional information; establishing the initial positional information as an origin in three-dimensional space for the initial anatomic image information; displaying a visual representation of the initial anatomic image information on a computerized display; receiving subsequent positional information from the sensor associated with movement of the patient; and updating the computerized display to reflect the subsequent positional information with respect to the initial positional information.

### BRIEF DESCRIPTION OF DRAWINGS

FIG. 1 is a block diagram view of an exemplar system and an associated patient and x-ray scanning apparatus.

FIG. 2 is a block diagram representation of components of the exemplary positional sensor.

FIG. 3 is a screen shot of a display provided by an exemplar system for total-hip-arthroplasty (THA).

FIG. 4 is a screen shot of a display provided by an exemplar system for THA.

FIG. 5 is a screen shot of a display provided by an exemplar system for THA.

FIG. 6 is a screen shot of a display provided by an exemplar system for total-knee-arthroplasty (TKA).

### DETAILED DESCRIPTION

The detailed description set forth below in connection with the appended drawings is intended as a description of exemplary embodiments of the current disclosure and is not intended to represent the only forms in which the embodiments may be constructed or utilized.

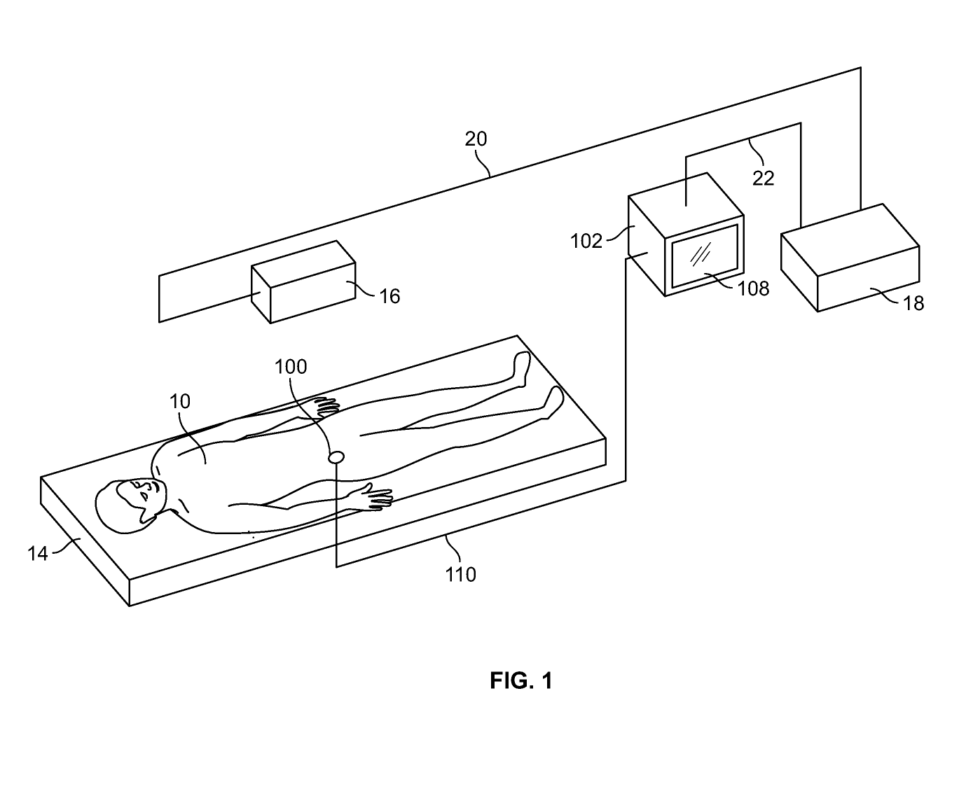

Referring to FIG. 1, a computerized visual orientation surgery assist computer 102 receives initial anatomic image information of a patient scan taken by an anatomical scanning device, such as an x-ray scanner 16, at a registration position of the patient 10 (lying on a patient table 14). The initial anatomic image information may be received from an image processing computer server 18 positioned via wired or wireless data links 20/22 between the x-ray scanner 16 and the surgery assist computer 102. The surgery assist computer 102 also receives initial positional information via wired or wireless data link 110 from a sensor 100 positioned/attached on the patient 10 at a registration position. The positional sensor 100 senses spatial position in three dimensions and transmits the positional information via the wired or wireless data link 110 to the surgery assist computer 102. The surgery assist computer 102 is programmed to establish the initial positional information as an origin in three-dimensional space for the initial anatomic image information; display a visual representation of the initial anatomic image information on a computerized display 108; receive subsequent positional information from the sensor 100 associated with movement of the patient 100; and update the computerized display 108 to reflect the subsequent positional information with respect to the initial positional information.

The computer 102 can have a receiver to receive the positional information via wired or wireless data link 110 from the positional sensor 100, a processor, such as a CPU, to process the positional information, a memory to store the positional information and any other information from the positional sensor 100, and a display 108 to display the orientation information to the surgeon and other healthcare providers.

Such a system (combination of the sensor 100 and surgery assist computer 102) may reduce the number of x-rays taken of a patient 10 during surgery by helping a surgeon identify desired orientation of the patient 10 via the computerized display 108 without having to take additional x-rays.

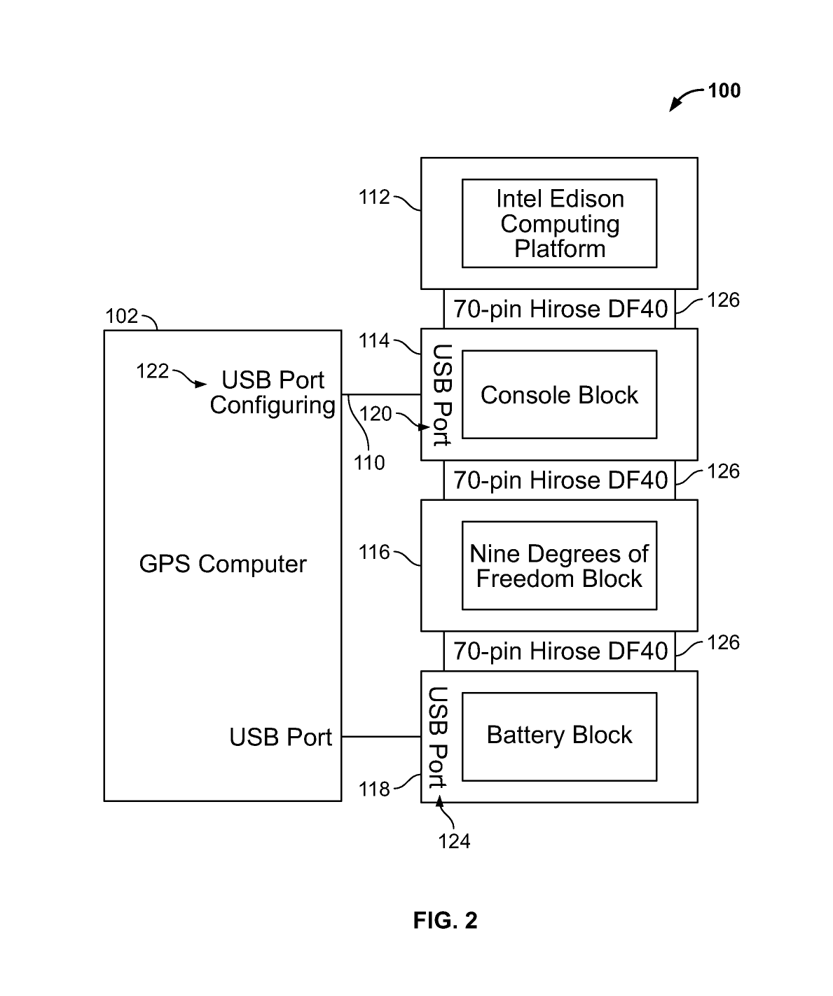

As shown in FIG. 2, an exemplary embodiment of the sensor 100 includes an Intel® Edison computing platform 112, a console block 114, a nine degree of freedom sensor block 116 and a battery block 118. In the exemplary embodiment, the blocks are individual circuit board assemblies stacked and connected via 70-pin Hirose DF40 connections 126. In such an embodiment, the sensor has dimensions of 1.79×1.22×0.78 inches. As shown in FIG. 2, the console block 114 includes a USB port 120 providing a wired USB connection 110 to a USB port 122 of computer 102 (which may be used to transmit positional information from the sensor 100 to the computer and/or be used to allow the computer 102 or another device to configure the sensor 100). The battery block 118 may be charged via a USB charging port 124 (which may or may not be the same as USB port 120 connected to computer 102). The Intel® Edison computing platform 112 hosts software that controls the nine degree of freedom sensors in sensor block 116 and collects data from the sensors. The nine degree of freedom sensor block contains a triple-axis gyrometer, a triple-axis accelerometer and a triple-axis magnetometer. The software in the Intel® Edison computing platform 112 utilizes a fusion algorithm to combine the outputs of the triple-axis gyrometer, the triple-axis accelerometer and the triple-axis magnetometer to generate positional information, such as pitch, yaw and roll information that can be sent/transmitted to the computer 102 over wired or wireless connection 110.

Typically, when the surgeon conducts a surgery, such as total hip arthroplasty (THA), the surgeon may position the patient accordingly and take an x-ray of the desired orientation. As the surgeon performs various steps of the surgery, the patient's body may be moved into various different positions. Eventually, during a certain portion of the surgery, the patient may need to be placed back in the desired orientation to complete a specific step in the surgical procedure, such as insertion of the acetabular component into the acetabulum. To assure that the patient is in the desired orientation, the surgeon may take another x-ray and compare the second x-ray to the first x-ray. The surgeon may repeat this process of taking additional x-rays until the desired orientation is achieved, thereby exposing the patient to harmful x-ray radiation each time.

Using the system (combination of the sensor 100 and surgery assist computer 102) can significantly reduce the x-ray radiation that the patient is exposed to during the surgery. The orientation sensor 100 can be positioned/attached to the patient 10 at a strategic location that allows the surgeon to identify the desired orientation of the patient 10, depending on the nature of the surgery. For example, for THA, the orientation sensor 100 can be placed on the iliac crest on the ipsilateral side of the surgery. Placing the orientation sensor 100 on the iliac crest allows the orientation sensor to monitor the necessary movement of the hip so as to track the anatomical part at issue (i.e. the acetabulum) without interfering with the surgery. The orientation sensor 100 may be temporarily fixed to the patient 10 with the use of adhesives or other types of fasteners that will allow the orientation sensor 100 to be removed when the surgery is complete.

Once the orientation sensor 100 is attached to the patient 10, the patient 10 is placed in the desired orientation. The orientation sensor 100 is configured to detect motion in three-dimensional space. Therefore, the orientation sensor 100 can detect tilting, rotation, and acceleration. For example, the orientation sensor 100 can detect tilting to the left and right (e.g. roll), or up and down (e.g. pitch). It can also detect rotational movement about a vertical axis (e.g. yaw).

Referring back to FIG. 1, once the orientation sensor 100 is attached and the patient 10 is in the desired initial orientation, the position of the orientation sensor 100 may be zeroed by the user of the computer 108; that is the user may activate a button, command or setting on the computer 102 to establish the initial position of the sensor 100 as an origin in three-dimensional space. As the patient 10 is moved about, the orientation sensor 100 monitors its movement and transmits its current orientation (relative to the initial position/orientation) to the computer 102 for display on the computerized display 108 as discussed below. When the surgeon reaches a step that requires the patient to be placed back into its initial orientation, the surgeon may monitor the display 108 and move the patient until the readings for the orientation sensor 100 are back at the origin in three-dimensional space (or at least back within a pre-set distance/orientation from the origin). In an embodiment, the computer 102 may be configured to emit visual and/or audible sounds and/or words to assist the practitioners with moving the patient 10 back to the initial orientation based upon positional information from the sensor 100. It is within the scope of the invention, therefore, that such origin setting and return-instruction functionality (or any other functionality described, herein, for the computer 102) can be integrated with the sensor 100.

If necessary or desired, a second intra-op x-ray may be taken to confirm that the patient is back to the registration or desired orientation. By using the orientation sensor 100 to place the patient so that the orientation sensor 100 is back in the zeroed position, the physician should be very close to, if not right on, the desired orientation. An intra-op X-ray can be taken to confirm. If the patient is still not exactly in the desired orientation, very little manipulation of the patient would be required to get the patient in the desired orientation. More so, multiple intra-op x-rays will not have to be taken to assure the desired orientation.

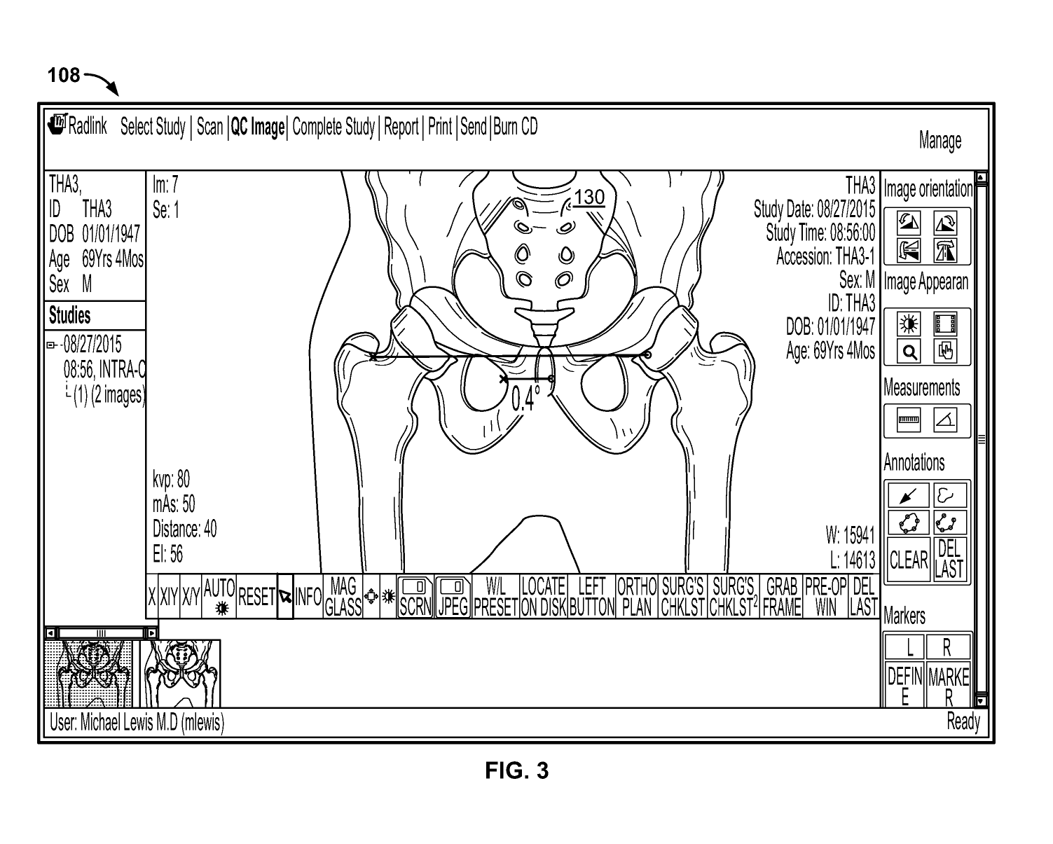

As shown in FIG. 3, in some embodiments, an initial pre-op x-ray of the pelvis 130 can be taken in the desired orientation and the position of the sensor 100 registered in the computer 102 as the initial/zeroed/origin for subsequent patient movements and sensed information from the sensor 100. Referring to FIG. 1, in an embodiment, to obtain this registration position, the x-ray emitter 16 is perpendicular to the floor (or perpendicular to the patient platform/bed 14). The x-ray emitter head 16 is set squarely in relation to the patient 10, in other words perpendicular to the body plane of the patient 10. In some embodiments, the x-ray image can be displayed on a grid to help identify the orientation of the pelvis. With a patient 10 in lateral position, and the FPD plate in level position, then the x-ray image vertical line is parallel to the floor/table 14 (or the x-ray horizontal line is perpendicular to the floor/table 14). Referring back to FIG. 3, then the transverse plane of the patient 10 can be derived by measuring the angle between the teardrop line and x-ray image horizontal line. Once this information is registered into the computer 102, the pitch readings of the sensor 100 can thereafter accurately tell its inclination against the patient's transverse plane. By attaching the sensor 100 (or a second orientation sensor 100) to an acetabula reamer, the computer 102 can guide the reaming for a targeted abduction angle based upon the position of the reamer-mounted sensor with respect to the registered origin and/or with respect to the patient-mounted sensor 100.

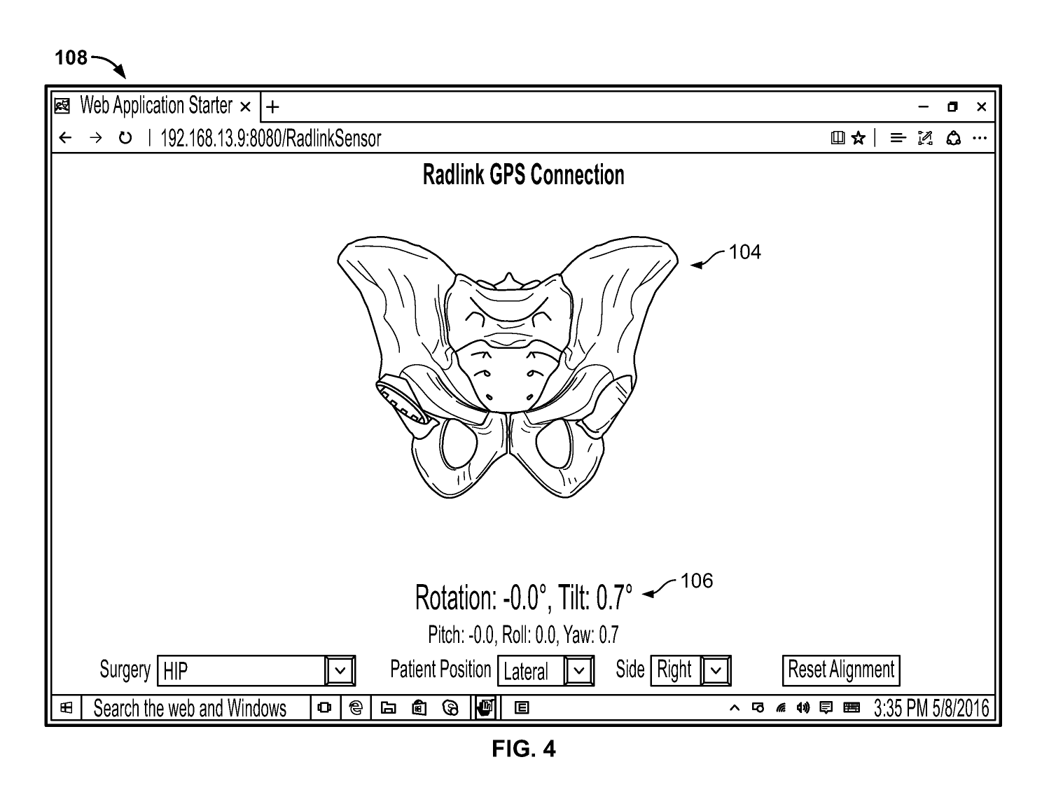

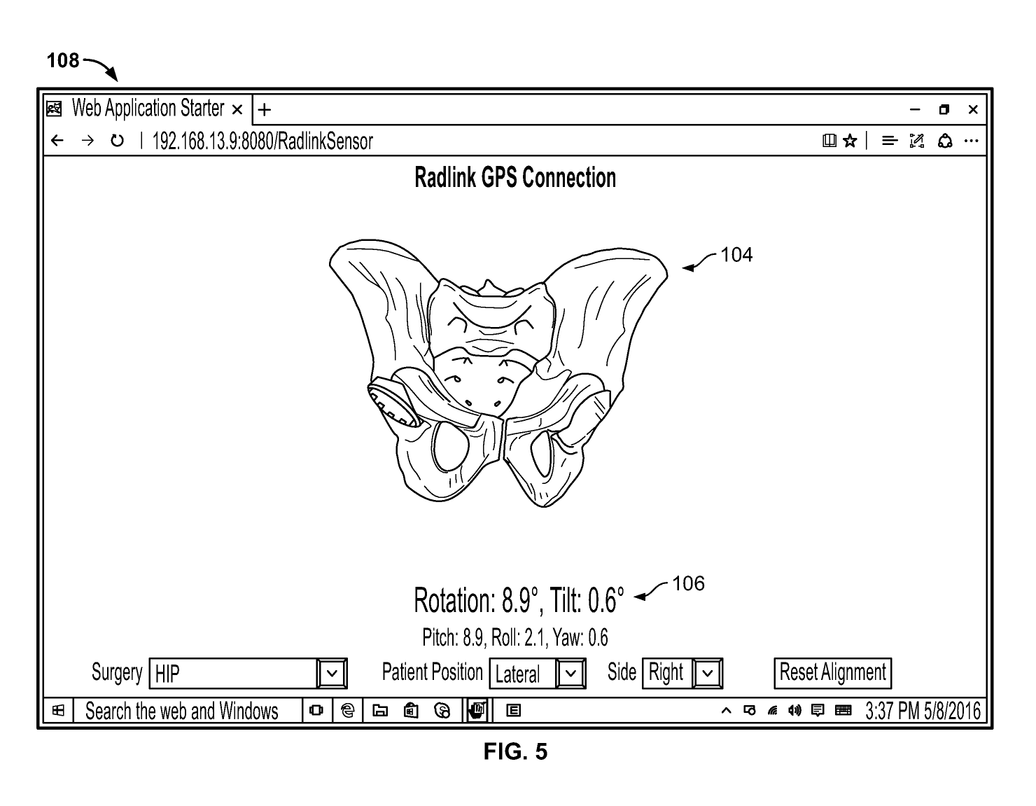

As shown in FIGS. 4 and 5, in some embodiments, with the sensor 100 attached to the patient's pelvis, x-ray images can be used (as discussed above) to register the patient's position into the computer 102 and/or with respect to the sensor 100. Once the x-ray image is shown on the display 108 to be in the desired registration position, the user may activate a button/command/link to inform the computer and/or the sensor 100 to zero out the sensor position as an origin in three-dimensional space. From this point on the computer 102 displays on the display 108 an animated/virtual image of the pelvis 104 that serves as a surrogate of the actual pelvis. As the patient's pelvis is moved and sensed by the sensor 100, the computer 102, receiving positional information from the sensor 100, moves the animated image 104 reflecting the two-dimensionally the pelvis position in three-dimensional space. This allows the visual depiction of the orientation of the virtual pelvis 104 to be registered to the orientation sensor 100 so that specific movement and readings on the orientation sensor 100 coordinate with the visual depiction of the orientation of the virtual pelvis 104 on the display 108 to represent actual movement of the pelvis in real time. As shown in FIGS. 4 and 5, the computer 102 may also display additional information 106 such as rotation and tilt readings of the patient's pelvis, as sensed by the sensor 100, with respect to the registration position.

In some total hip arthroplasty procedures, the acetabular cup position as measured on radiographic X-ray image is not accurate when the patient's pelvis is tilted (Sagittal Plane) and/or rotated (Transverse plane). Adjustment factors are needed to compensate the non-ideal patient orientation, such that no additional X-rays are required to derive more accurate measurements for both abduction and anteversion.

Acetabular cup abduction and anteversion adjustment factors' calculation is based on the study of a projected circle in 3-dimensional space. The rotation of the circle in 3-dimensional space mimics the rotation of acetabular cup. An acetabular cup will display shapes of ellipses under different angle of projections. There are three rotation factors that will affect the shape of the projected ellipse. The three rotation factors are Abduction (I)—rotation around Z axis, Anteversion (A)—rotation around Y axis, Tilt (T)—rotation around X axis. At the end of the three rotations, a projected ellipse will be shown on an X-Y plane.

Applying 3 rotations on a circle will result in a similar effect. The equation of the circle after three rotations is:

X=R*[sin(θ)*cos(I)*cos(A)+cos(θ)*sin(A)]Y=R*cos(T)*sin(θ)*sin(I)−R*[−sin(θ)*cos(I)*sin(A)*sin(T)+cos(θ)* cos(A)*sin(T)]Y=R*cos(T)*sin(θ)*sin(I)−R[−sin(θ)*cos(I)*sin(A)*sin(T)+cos(θ)*cos(A)*sin(T)]

where X and Y represent the coordinates of the projected ellipse on the X-Y plane, R represents the size of the cup, and θ represents the parameter.

The equation of the normal of the circle surface after three rotations is:

Xₙₒᵣₘₐₗ=sin(I)*cos(A) Yₙₒᵣₘₐₗ=cos(I)*cos(T)+sin(I)*sin(A)*sin(T)

The projected ellipse abduction angle and major/minor diameter of the ellipse at different orientation can be calculated based on the above equations. Conversely, using the same method, we could use the measurement from radiographic images to reverse calculate the orientation of the acetabular cup.

Assuming we have a way to determine the pelvic tilt and rotation, we can further calculate the true orientation of the cup, thus derive the adjustment factors for abduction and anteversion.

Another problem involves how to determine the pelvic tilt and rotation when the X-ray is taken. In one embodiment, the pelvic tilt can be estimated by measuring the pelvic ratios from the pre-op and intra-op X-rays. The pelvic rotation can be estimated by measuring distance between mid-sacrum line and mid-symphysis line on the intra-op X-ray and comparing the distance to the previous distance of the same landmarks in the pre-op X-ray.

In another embodiment, as discussed above, before the surgery starts, the orientation sensor 100 can be attached on the patient's iliac crest. The orientation sensor 100 is calibrated to align the sensor's axis with the patient's anatomic axis. X-ray may be used to confirm that the patient orientation matches the pre-op X-ray. At this point, the orientation sensor 100 may be reset to mark the zero position. When the intra-op X-ray is taken, the orientation sensor's read out includes both pelvic tilt and rotation.

Another problem encountered in hip surgery is that the cup position, as measured on radiographic X-ray image, is not accurate when the patient's pelvis is tilted (Sagittal Plane) and/or rotated (Transverse plane). A way to ensure perfect patient orientation without extra X-rays is needed to guide the repositioning of the patient.

In one embodiment, before the surgery starts, the orientation sensor 100 may be attached on the patient's iliac crest. X-ray may be used to confirm on the display 108 that the patient orientation matches the pre-op X-ray. At this point, the orientation sensor 100 is reset to mark the zero position. After interim surgical steps are performed, when the patient is ready to be placed back into the desired orientation, the patient is repositioned such that the orientation sensor shows its zero position before an intra-op X-ray is taken. This maximizes the assurance that the patient is in the desired orientation.

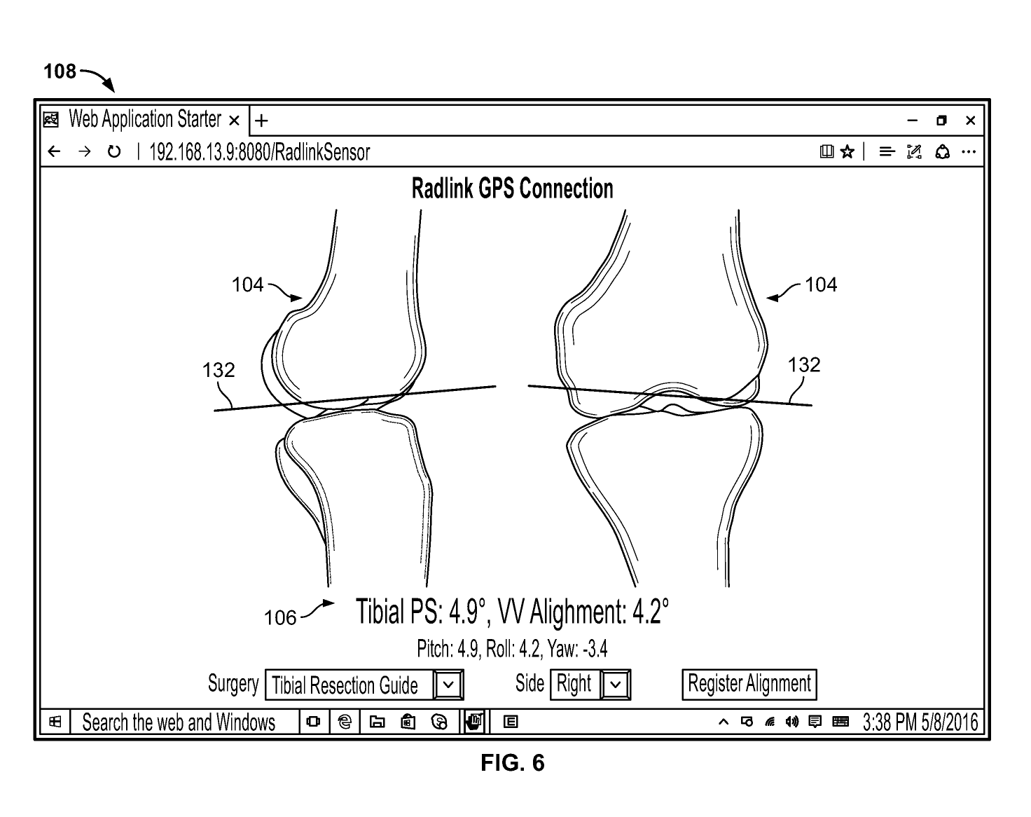

As shown in FIG. 6, the sensor 100 and associated computer 102 and display 108 may be used as a total-knee-arthroplasty (TKA) cutting guide. With the sensor 100 attached to TKA cutting block jigs, an AP and a lateral x-ray image can be used to register the cutting block orientation into the computer 102 and/or sensor 100. Once registered, the computer 102 displays reference lines 132 that reflect the cutting block's posterior slope and valgus/varus alignment scope with respect to animated/virtual images 104 of the patient's knee along with positional information 106 of the sensor 100 with respect to the registration position or origin.

While the above embodiments have been described with respect to THA and TKA procedures, it should be appreciated that the current disclosure is not limited for use with such procedures and other uses may fall within the scope of the current disclosure. For example, and without limitation, the sensor 100 and computer 102 may be used for bone prep measurements, orienting implant placement tools (e.g., mounting to instruments as described above to help guide the instruments during a procedure), stitching procedures, fracture fixation, ankle procedures, spinal procedures, and the like.

As another example, the sensor 100 and computer 102 may be used to sense and display positional information pertaining to fibular apex in relation to tibial cortex as an indicator of “neutral AP rotation;” where such orientation information would allow verification of cutting tool position to permit a surgeon to reproducibly create the desired femoral component rotation in TKA.

To provide additional context for the computer 102, the following discussion is intended to provide a brief, general description of a suitable computing environment in which the various aspects of the disclosure may be implemented. While some exemplary embodiments of the disclosure relate to the general context of computer-executable instructions that may run on one or more computers, those skilled in the art will recognize that the disclosure also may be implemented in combination with other program modules and/or as a combination of hardware and software. An exemplary embodiment of the computer 102 may include a computer that includes a processing unit, a system memory and a system bus. The system bus couples system components including, but not limited to, the system memory to the processing unit. The processing unit may be any of various commercially available processors. Dual microprocessors and other multi-processor architectures may also be employed as the processing unit.

The system bus may be any of several types of bus structure that may further interconnect to a memory bus (with or without a memory controller), a peripheral bus, and a local bus using any of a variety of commercially available bus architectures. The system memory may include read only memory (ROM) and/or random access memory (RAM). A basic input/output system (BIOS) is stored in a non-volatile memory such as ROM, EPROM, EEPROM, which BIOS contains the basic routines that help to transfer information between elements within the computer, such as during start-up. The RAM may also include a high-speed RAM such as static RAM for caching data.

The computer 102 may further include an internal hard disk drive (HDD) (e.g., EIDE, SATA), which internal hard disk drive may also be configured for external use in a suitable chassis, a magnetic floppy disk drive (FDD), (e.g., to read from or write to a removable diskette) and an optical disk drive, (e.g., reading a CD-ROM disk or, to read from or write to other high capacity optical media such as the DVD). The hard disk drive, magnetic disk drive and optical disk drive may be connected to the system bus by a hard disk drive interface, a magnetic disk drive interface and an optical drive interface, respectively. The interface for external drive implementations includes at least one or both of Universal Serial Bus (USB) and IEEE 1394 interface technologies.

The drives and their associated computer-readable media may provide nonvolatile storage of data, data structures, computer-executable instructions, and so forth. For the computer, the drives and media accommodate the storage of any data in a suitable digital format. Although the description of computer-readable media above refers to a HDD, a removable magnetic diskette, and a removable optical media such as a CD or DVD, it should be appreciated by those skilled in the art that other types of media which are readable by a computer, such as zip drives, magnetic cassettes, flash memory cards, cartridges, and the like, may also be used in the exemplary operating environment, and further, that any such media may contain computer-executable instructions for performing the methods and processes of the current disclosure.

A number of program modules may be stored in the drives and RAM, including an operating system, one or more application programs, other program modules and program data. All or portions of the operating system, applications, modules, and/or data may also be cached in the RAM. It is appreciated that the invention may be implemented with various commercially available operating systems or combinations of operating systems.

It is within the scope of the disclosure that a user may enter commands and information into the computer through one or more wired/wireless input devices, for example, a touch screen display, a keyboard and/or a pointing device, such as a mouse. Other input devices may include a microphone (functioning in association with appropriate language processing/recognition software as known to those of ordinary skill in the technology), an IR remote control, a joystick, a game pad, a stylus pen, or the like. These and other input devices are often connected to the processing unit through an input device interface that is coupled to the system bus, but may be connected by other interfaces, such as a parallel port, an IEEE 1394 serial port, a game port, a USB port, an IR interface, etc.

A display monitor 108 or other type of display device may also be connected to the system bus via an interface, such as a video adapter. In addition to the monitor, a computer may include other peripheral output devices, such as speakers, printers, etc.

The computer 102 may operate in a networked environment using logical connections via wired and/or wireless communications to one or more remote computers. The remote computer(s) may be a workstation, a server computer, a router, a personal computer, a portable computer, a personal digital assistant, a cellular device, a microprocessor-based entertainment appliance, a peer device or other common network node, and may include many or all of the elements described relative to the computer. The logical connections depicted include wired/wireless connectivity to a local area network (LAN) and/or larger networks, for example, a wide area network (WAN). Such LAN and WAN networking environments are commonplace in offices, and companies, and facilitate enterprise-wide computer networks, such as intranets, all of which may connect to a global communications network such as the Internet.

The computer 102 may be operable to communicate with any wireless devices or entities operatively disposed in wireless communication, e.g., the position sensor 100, a printer, scanner, desktop and/or portable computer, portable data assistant, communications satellite, any piece of equipment or location associated with a wirelessly detectable tag (e.g., a kiosk, news stand, restroom), and telephone. This includes at least Wi-Fi (such as IEEE 802.11x (a, b, g, n, etc.)) and Bluetooth™ wireless technologies. Thus, the communication may be a predefined structure as with a conventional network or simply an ad hoc communication between at least two devices.

The computer 102 may be any type of computing device or system available; including, without limitation, one or more desktop computers, one or more server computers, one or more laptop computers, one or more handheld computers, one or more tablet computers, one or more smartphones, one or more cloud-based computing systems, one or more wearable computers, and/or one or more computing appliances and the like.

While exemplary embodiments have been set forth above for the purpose of disclosure, modifications of the disclosed embodiments as well as other embodiments thereof may occur to those skilled in the art. Accordingly, it is to be understood that the disclosure is not limited to the above precise embodiments and that changes may be made without departing from the scope. Likewise, it is to be understood that it is not necessary to meet any or all of the stated advantages or objects disclosed herein to fall within the scope of the disclosure, since inherent and/or unforeseen advantages of the may exist even though they may not have been explicitly discussed herein.

What is claimed is:

1. A visual orientation surgery assist system comprising:
a positional sensor sensing spatial position in three dimensions and transmitting positional information in three dimensions; and
a computerized display system including a display, a receiver receiving the positional information from the positional sensor, a microcontroller operatively coupled to the receiver and to the display and having access to system memory, the system memory including software instructions causing the CPU to perform the steps of;
receiving initial anatomic image information of a patient scan taken at a registration position of the patient;
receiving initial positional information from the sensor positioned on the patient at the registration position;
establishing the initial positional information as an origin in three-dimensional space for the initial anatomic image information;
displaying a visual representation of the initial anatomic image information on the display;
receiving subsequent positional information from the sensor associated with movement of the patient; and
updating the display to reflect the subsequent positional information with respect to the initial positional information.

2. The visual orientation surgery assist system of claim 1, wherein the positional sensor includes a triple-axis gyrometer, a triple-axis accelerometer, and a triple-axis magnetometer.

3. The visual orientation surgery assist system of claim 2, wherein the positional sensor further includes a computing component programmed with a fusion algorithm that combines outputs of the triple-axis gyrometer, the triple-axis accelerometer, and the triple-axis magnetometer into positional information comprising pitch, yaw and roll information.

4. The visual orientation surgery assist system of claim 1, wherein the positional information transmitted by the positional sensor includes pitch, yaw and roll information.

5. The visual orientation surgery assist system of claim 1, wherein the patient scan includes an x-ray scan.

6. The visual orientation surgery assist system of claim 5, wherein the visual representation of the initial anatomic image information on the display includes x-ray scan images.

7. The visual orientation surgery assist system of claim 6, wherein the subsequent positional information updated to the display includes tilt and rotation information overlayed with the visual representation of the initial anatomic image information.

8. The visual orientation surgery assist system of claim 6, wherein the subsequent positional information updated to the display includes translational information with respect to the origin overlayed with the visual representation of the initial anatomic image information.

9. The visual orientation surgery assist system of claim 8, wherein the software instructions cause the CPU to perform the additional step of providing at least one of a visual and an audible notification when the subsequent positional information updated to the display reaches a predetermined proximity to the origin.

10. The visual orientation surgery assist system of claim 6, wherein the subsequent positional information updated to the display includes reference lines reflecting updated orientations for surgical procedural steps overlayed with the visual representation of the initial anatomic image information.

11. The visual orientation surgery assist system of claim 6, wherein the subsequent positional information updated to the display includes reference ellipses reflecting updated orientations for surgical procedural steps overlayed with the visual representation of the initial anatomic image information.

12. The visual orientation surgery assist system of claim 1, wherein the visual representation of the initial anatomic image information on the display includes an animated virtual representation of an anatomical body part associated with the location of the positional sensor on the patient's anatomy.

13. The visual orientation surgery assist system of claim 12, wherein the subsequent positional information updated to the display includes animation of the virtual representation of the anatomical body part.

14. The virtual orientation surgery assist system of claim 13, wherein the animation of the virtual representation of the anatomical body part includes animations representing movement of the anatomical body part in three-dimensional space.

15. The virtual orientation surgery assist system of claim 14, wherein the animation of the virtual representation of the anatomical body part includes two-dimensional animations representing movement of the anatomical body part in three-dimensional space.

16. The virtual orientation surgery assist system of claim 14, wherein the animation of the virtual representation of the anatomical body part includes an animated representation of a surgical implant implanted thereto.

17. The virtual orientation surgery assist system of claim 14, wherein the animation of the virtual representation of the anatomical body part includes an animated representation of a surgical tool associated therewith.

18. The virtual orientation surgery assist system of claim 14, wherein the animation of the virtual representation of the anatomical body part includes a representation of surgical steps to be performed with respect to the anatomical body part.

19. The virtual orientation surgery assist system of claim 18, wherein the representation of surgical steps to be performed with respect to the anatomical body part is an animated representation of surgical steps that represent movement of aspects of the surgical step in three-dimensional space as the anatomical body part is moved.

20. A computerized visual orientation surgery assist method comprising steps of:
receiving initial anatomic image information of a patient scan taken at a registration position of the patient;
receiving initial positional information from a sensor positioned on the patient at a registration position, the positional sensor sensing spatial position in three dimensions and transmits the positional information;
establishing the initial positional information as an origin in three-dimensional space for the initial anatomic image information;
displaying a visual representation of the initial anatomic image information on a computerized display;
receiving subsequent positional information from the sensor associated with movement of the patient; and
updating the computerized display to reflect the subsequent positional information with respect to the initial positional information.

21. The method of claim 20, wherein the step of displaying a visual representation of the initial anatomic image information on the display includes displaying x-ray scan images.

22. The method of claim 21, wherein the step of updating the computerized display includes displaying tilt and rotation information overlayed with the visual representation of the initial anatomic image information.

23. The method of claim 21, wherein the step of updating the computerized display includes displaying translational information with respect to the origin overlayed with the visual representation of the initial anatomic image information.

24. The method of claim 23, further comprising a step of providing at least one of a visual and an audible notification when the subsequent positional information updated to the display reaches a predetermined proximity to the origin.

25. The method of claim 21, wherein the step of updating the computerized display includes displaying reference lines reflecting updated orientations for surgical procedural steps overlayed with the visual representation of the initial anatomic image information.

26. The method of claim 21, wherein the step of updating the computerized display includes displaying reference ellipses reflecting updated orientations for surgical procedural steps overlayed with the visual representation of the initial anatomic image information.

27. The method of claim 20, wherein the step of displaying a visual representation of the initial anatomic image information on the display includes displaying an animated virtual representation of an anatomical body part associated with the location of the positional sensor on the patient's anatomy.

28. The method of claim 27, wherein the step of updating the computerized display includes displaying animation of the virtual representation of the anatomical body part.

29. The method of claim 28, wherein the animation of the virtual representation of the anatomical body part includes animations representing movement of the anatomical body part in three-dimensional space.

30. The method of claim 29, wherein the animation of the virtual representation of the anatomical body part includes two-dimensional animations representing movement of the anatomical body part in three-dimensional space.

31. The virtual orientation surgery assist system of claim 29, wherein the animation of the virtual representation of the anatomical body part includes an animated representation of a surgical implant implanted thereto.

32. The virtual orientation surgery assist system of claim 29, wherein the animation of the virtual representation of the anatomical body part includes an animated representation of a surgical tool associated therewith.

33. The virtual orientation surgery assist system of claim 29, wherein the animation of the virtual representation of the anatomical body part includes a representation of surgical steps to be performed with respect to the anatomical body part.

34. The virtual orientation surgery assist system of claim 33, wherein the representation of surgical steps to be performed with respect to the anatomical body part is an animated representation of surgical steps that represent movement of aspects of the surgical step in three-dimensional space as the anatomical body part is moved.
